(19) United States
(12) Patent Application Publication
(10) Pub. No.: US 2005/0267453 A1
(43) Pub. Date: Dec. 1, 2005

(54) High intensity focused ultrasound for imaging and treatment of arrhythmias

(76) Inventors: Serena H. Wong, Menlo Park, CA (US); David H. Liang, Menlo Park, CA (US); Girish Narayan, Sunnyvale, CA (US)

Correspondence Address: LUMEN INTELLECTUAL PROPERTY SERVICES, INC., 2345 YALE STREET, 2ND FLOOR, PALO ALTO, CA, 94306, US

(21) Appl. No.: 11/137,961

(22) Filed: May 25, 2005

Related U.S. Application Data
(60) Provisional application No. 60/575,455, filed on May 27, 2004.

(51) Int. Cl. A61B 18/04; A61B 8/00

(52) U.S. Cl. 606/27; 600/439

(57) ABSTRACT

A dual-mode, capable of imaging and ablation, ultrasound array integrated in a catheter is provided for minimally invasive treatment of arrhythmias. The catheter array is small enough for insertion through a peripheral vein and is longitudinally integrated with the catheter to have a side-view for tissue imaging and ablation. A high length/width array ratio creates a large aperture necessary for high power ablation densities. A catheter stabilization device maintains a distance between the catheter array and the wall of a heart or vein. Visualization of anatomy and imaging of ablated tissue provides a guide for placing the lesion and assists in achieving a pattern of ablated tissue. The catheter is advanced to another area by catheter rotation and/or array steering or focusing. Gating the imaging and ablation processes to a heart cycle allows for accounting and compensation of heart motion and enables automation of an arrhythmia treatment.

### CROSS-REFERENCE TO RELATED APPLICATIONS

The present application claims priority from U.S. Provisional Patent Application with No. 60/575,455 filed on May 27, 2004, the entire content of which is incorporated herein by reference.

### FIELD OF THE INVENTION

The present invention relates generally to treatments of arrhythmias. More particularly the invention relates to intravascular imaging and treatment of atrial fibrillation with a single dual mode high intensity focused ultrasound array.

### BACKGROUND

A large percentage of the US population suffers from arrhythmias, irregular contractions of the heart that degrade the quality of life and increases mortality and morbidity. In particular, atrial fibrillation (AF) affects about 1% of the population and is responsible for 15-20% of all strokes; this results in more than 460,000 hospitalizations ($2.8 billion from hospitalizations alone) per year.

AF is caused by the breakdown of ordered electrical propagation within the atrium into a chaotic series of circulating wavefronts. These chaotic wavefronts are frequently initiated by foci of activity originating from the pulmonary veins. These unsynchronized contractions of the atria (about 300 beats per minute) reduce cardiac output. This degrades a patient's quality of life and can lead to clot formation followed by strokes and other suffering.

AF has commonly been treated by anti-arrhythmia drugs, which organize the contractions of the atria. Alternative treatments are surgical (e.g. Cox MAZE procedures) procedures that isolate aberrant electrical foci and circular conduction pathways. Electrically isolating and ablating responsible foci with appropriately placed lesions has been proven to be effective in curing arrhythmias and substantially reducing healthcare utilization.

However, widespread acceptance of ablative strategies for curing atrial fibrillation has been limited by the need for surgical intervention and the difficulty and limited success of catheter based approaches. Long-term success with catheter-based ablations remains limited to about b 80%, despite the need for more than 1.5 procedures on average per patient. The catheter-based procedures also remain tedious with procedure times of over 7 hours.

Some of the challenges of catheter ablation are visualization and mechanical positioning of the catheter tip. Visualization catheter procedures remain dependent upon fluoroscopy for visualization, which produces 2-D projection images of the chest with little tissue contrast. The structures and anatomy of the heart relative to the position of the catheters are hard to determine, so it is difficult to direct the catheter to a specific site for ablation. Second, once a lesion has been created, this location cannot be visualized, which makes it difficult to determine where to place neighboring lesions to create a continuous burn. Furthermore, because catheter procedures are so time consuming, they result in extensive radiation exposure.

Mechanical control and positioning remain difficult because of the mechanics of the catheter itself and the physiology of the heart. In most cases, the treatment electrode or device is located at the end of a 1-meter flexible shaft. Position control is limited to rotations of the catheter shaft and deflection of the catheter tip. With this crude control, revisiting previous treatments spots or moving to specific neighboring locations to produce a continuous lesion is also difficult. The variable anatomy of the region and motion of the heart make the task more difficult. Ablation inside the vein often causes restenosis, which causes respiratory symptoms such as shortness of breath, cough, hemoptysis, and in more cases may lead to pneumonia.

The optimal burn is placed at the ostia to the veins. However, vein size (15 mm diameter plus or minus 10 mm), angulation (plus or minus 15 degrees), and/or vein geometries are highly variable from person to person, making placement difficult. Heart motion also makes the task more difficult. Therefore it would be desired to have lesion formation occur quickly during the heart cycle or have lesion formation gated between heart cycles to minimize effects from heart motion. Accordingly, it is considered an advance in the art to develop new techniques to perform more effective and efficient procedures.

### SUMMARY OF THE INVENTION

The present invention provides an apparatus and a method for minimally invasive treatment of arrhythmias such as atrial fibrillation. In this invention, a dual-mode high intensity focused ultrasound array is integrated with a catheter. The catheter array is small enough in diameter to be inserted through a peripheral vein such as the femoral vein or internal jugular vein. The array is a 1-D array or a 2-D array of 32 or more dual-mode ultrasound transducer elements (capable of both ablation and imaging) and is integrated in the longitudinal direction of the catheter. This longitudinal design provides a side-view with respect to a heart wall or a wall of a vein.

The dual modes of the array are imaging a region of interest and ablating a treatment area that is within the region of interest. The array has a high length to width ratio to achieve a large aperture, which is necessary for high power densities in ablation mode. It is preferred to achieve a power density at the focal area or point of a treatment area of at least 200 W/cm². The high length/width ratio further achieves tissue ablation in the form of knife-cuts, i.e. more or less linear or ellipsoidal burns/lesions. These knife-cuts allow for the creation of a pattern, preferably a continuous pattern, of ablated tissue.

A stabilization device for the catheter is preferred to stabilize the device within the moving heart and to maintain a distance between the catheter array and the wall of the heart or a vein. For example the stabilization device is positioned near or in the pulmonary vein of the heart when imaging and treating atrial fibrillation. By physically fixing the array at a distance from the heart wall, the array can image the region of interest from afar. With this information, difficulties in variable anatomy and motion between heart cycles can be compensated for by dynamically, electronically focusing the therapy to the treatment area.

A processing means includes the necessary, electronics/hardware, software and algorithms to handle and control the imaging and ablation. In one aspect of the invention the sequence of events could be as follows. An image is obtained of a region of interest in imaging mode. The obtained image is then evaluated and a treatment area is determined. For this treatment area ablation parameters are set after which the elements in the array are adjusted to focus and deliver the treatment to the treatment area in ablation mode. The high power density allows the visualization and imaging of ablated tissue, which can be used to guide the system or user to a next treatment area and achieve a continuous area of ablated tissue over the wall of a vein. The catheter can be advanced to another treatment area by either rotating the catheter to a new area and/or steering the array to that new area.

The processing means can be gated to the heart cycle or part of the heart cycle. Gating could be established by a gating processor or a heart cycle monitoring means. In one example, the heart cycle is monitored to coincide with the timing and execution of the processing means within a time frame of a heart cycle, e.g. atrial diastole. Gating provides the opportunity to compensate and account for heart motion as well as automates the imaging and ablation process and significantly reduces the overall treatment time.

### BRIEF DESCRIPTION OF THE DRAWINGS

The objectives and advantages of the present invention will be understood by reading the following detailed description in conjunction with the drawings, in which:

FIG. 1 shows a catheter with a high intensity focused ultrasound transducer array in longitudinal position and a side-view for imaging and treatment according to the present invention.

FIG. 2 shows a catheter according to FIG. 1 with a field of view for imaging a region of interest (top) and with a focused field of view for ablating a treatment area (bottom), both according to the present invention.

FIG. 3 shows a processing means to drive the dual mode high intensity focused ultrasound transducer array. The array is switched between the imaging and ablation electronics. When attached to the imaging electronics, scan images are acquired. This data is then sent to the computer where post-processing and lesions detection algorithms detect the location of anatomy and/or previous burns. Then the operator or the computer can select a location for the next burn. The software calculates and programs the parameters necessary to focus the therapy at the desired location.

FIG. 4 shows exemplary array designs according to the present invention.

FIG. 5 shows an example of a time sequence for gating the imaging and ablation sequence according to the present invention.

### DETAILED DESCRIPTION

Although the following detailed description contains many specifics for the purposes of illustration, anyone of ordinary skill in the art will readily appreciate that many variations and alterations to the following exemplary details are within the scope of the invention. Accordingly, the following preferred embodiments of the invention are set forth without any loss of generality to, and without imposing limitations upon, the claimed invention.

The apparatus of the present invention is a dual-mode high intensity focused ultrasound array 110 with an ablation mode and an imaging mode. Array 110 is integrated in the longitudinal direction of a catheter 120 and has a side-view with respect to catheter 120. In one aspect, the diameter of the catheter is up to 4 mm and is preferably 2-3 mm. In yet another aspect, the catheter is a 7-French catheter with a diameter of about 2.31 mm. The length of the array should be at least 15 mm and is preferably about 20-30 mm. The width of the array should be up to 3 mm and could be flat or curved. The array could be a 1-D or 2-D array of 32 or more dual-mode ultrasound transducer elements as long as the size constraints indicated herein are maintained.

One of the reasons for these size constraints of the catheter and the array is that the apparatus should be small enough to be inserted through a peripheral vein such as the femoral vein or internal jugular vein. For example, in one aspect of the invention the catheter array is used for imaging and treatment of an atrial fibrillation. The catheter is then inserted through one of these peripheral veins to the left atrium 130 and positioned with the tip of catheter 120 near or in the pulmonary vein 140. Yet another reason for the size constraints and placement of the array in longitudinal direction is that it is the objective of the invention to ablate a large area or line (i.e. treatment area 230). Additionally, it is the objective to form knife-cuts (i.e. more or less linear or ellipsoidal burns/lesions) into a continuous pattern. To achieve this aspect of the invention, the array is defined with a high length/width aspect ratio. In one aspect, the length/width ratio of the array is at least 5 (e.g. 15 mm by 3 mm). In another aspect, the length/width ratio is at least 15 (e.g. 30 mm by 2 mm).

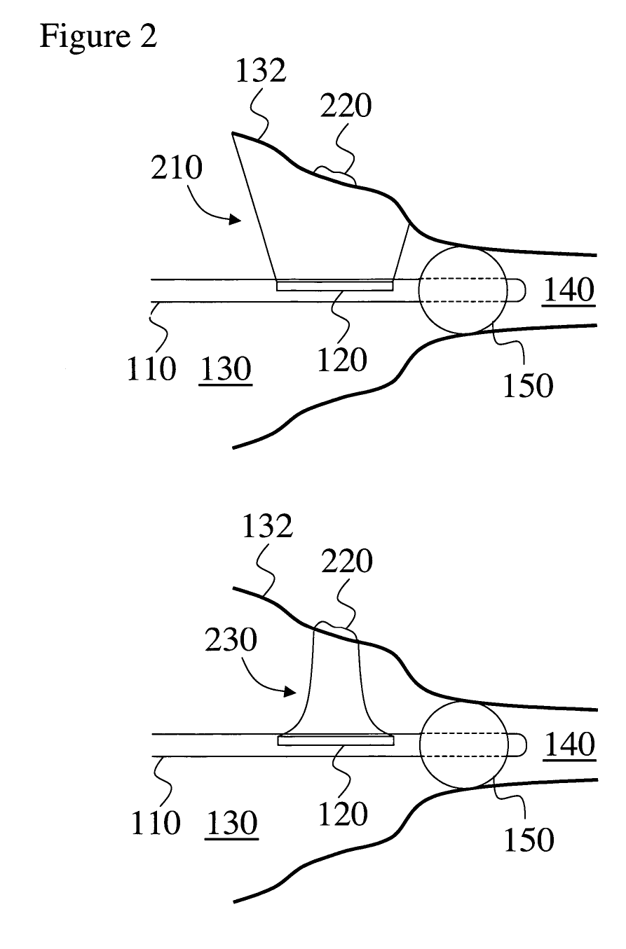

The high length/width aspect ration further enables an array with a large aperture, which is necessary to achieve the high power densities for ablation. These power densities should be at least 200 W/cm² at the focal area/point of treatment area 230 within a region of interest 220. Defined differently, these power density should be at least enough to establish tissue necrosis which is achieved when temperatures at 43 C or higher are achieved at the focal area/point. The power density should also be high enough to ablate in a short time frame, e.g. within a heart cycle or within part of a heart cycle. Yet another reason for the power density to be high is to be able to visualize and image the ablated tissue to guide the system or user to a next treatment area and achieve a continuous area over the wall of a vein. Another reason for the size constraints is to obtain an image field of view 210 of a region of interest 220 for imaging with a distance of at least 5-20 mm between the array and the wall 132 of heart 130 or the wall of pulmonary vein 140 (See FIG. 2). With such an area, it is possible to produce continuous patterns over the wall, e.g. pulmonary vein.

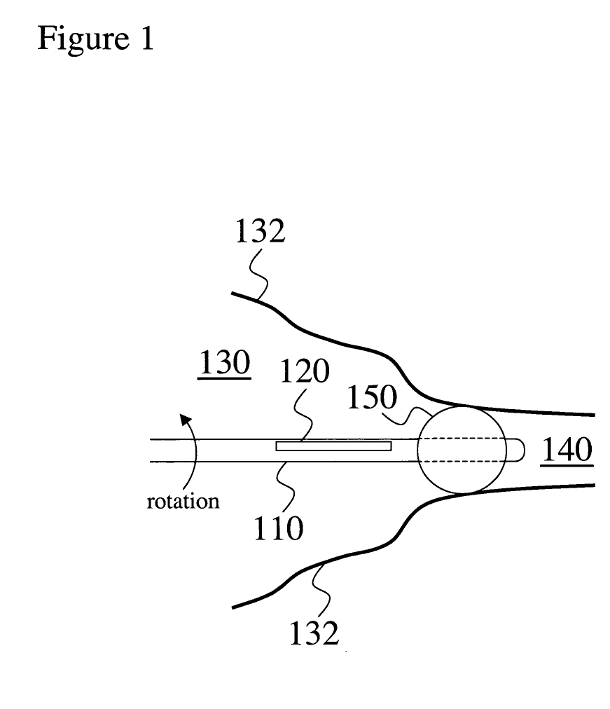

A stabilization device anchors the catheter inside the heart. By physically fixing the array at a distance from the heart wall, the array can image the region of interest from afar. With this information, difficulties in variable anatomy and motion between heart cycles can be compensated for by dynamically, electronically focusing the treatment to the treatment area 220. In a specific example, a stabilization device 150 could be added to catheter 120 to stabilize the array 110 within the left atrium 130 at a distance from the wall 132 or pulmonary vein 140. Examples of such a stabilization device are e.g. a balloon, a hollow balloon, a mesh, a legged device, or the like as they are known in the art. In the example of FIGS. 1-2 a balloon is positioned near the tip of the catheter and positioned near or in pulmonary vein 140.

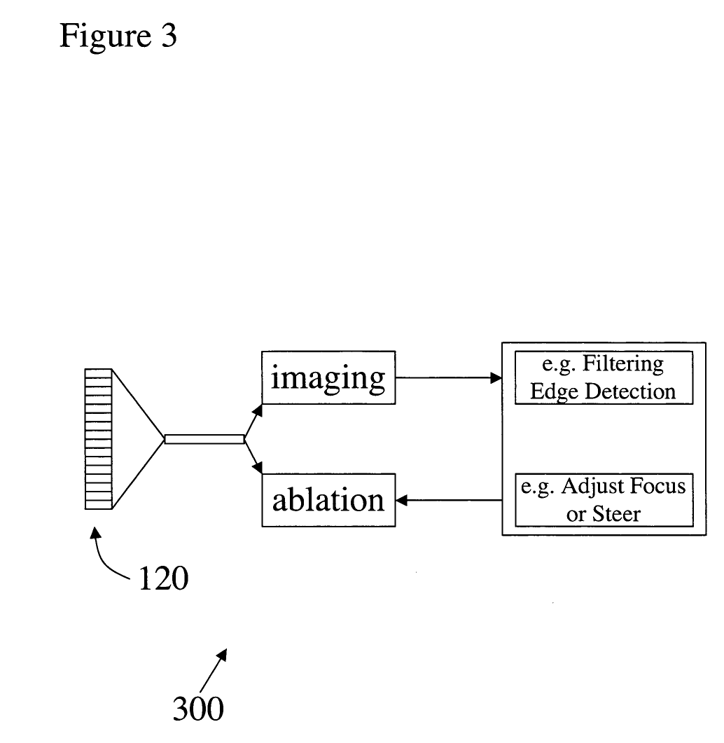

The apparatus further includes a processing means 300 (See FIG. 3), which includes all the necessary electronics, hardware as well as computer hardware and software to handle and control the imaging and ablation steps. The transducer array 120 can be switched between imaging and ablation electronics. Data from the imaging electronics is sent to a computer; post processing and image formation algorithms are executed to determine the lesion location. The focus will be adjusted and the ablation electronics will be programmed. In one aspect the sequence could be as follows. Once the catheter is positioned near a region of interest an image scan 210 is obtained of the region of interest in the imaging mode (See FIGS. 2-3). For imaging, a simple B-scan, sector-scan, or synthetic aperture can be used. The imaging electronics can be implemented as either a series of multiplexed pulser and receivers (for B-scan, sector scan, or synthetic aperture) or as a full phased array as known in the art. The image is processed (e.g. filtering and edge detection) to evaluate the obtained image and determine a treatment area 220. Ablation parameters (e.g. apodization, focus, and steering) for treatment area 220 are determined using phase-array techniques known in the art. The elements in the array are then focused and steered 230 to treatment area 220, which is then ablated in ablation mode. Examples of ablation parameters are: 1) >0.25 W output per channel at frequencies above 1 MHz to achieve the necessary power density at the focus, 2) <1 radian phase steps for beam forming, and 3) magnitude scaling to compensate for inconsistencies in the channels and for grating lobe reduction algorithms.

Once a lesion (burn) is created, an image of the lesion could be obtained and used for determining the next treatment area. This would assist in developing a pattern or continuous lesion over the wall of the vein. In advancing the catheter to another region of interest one could either rotate the catheter to a new region and/or steer the array to that new region.

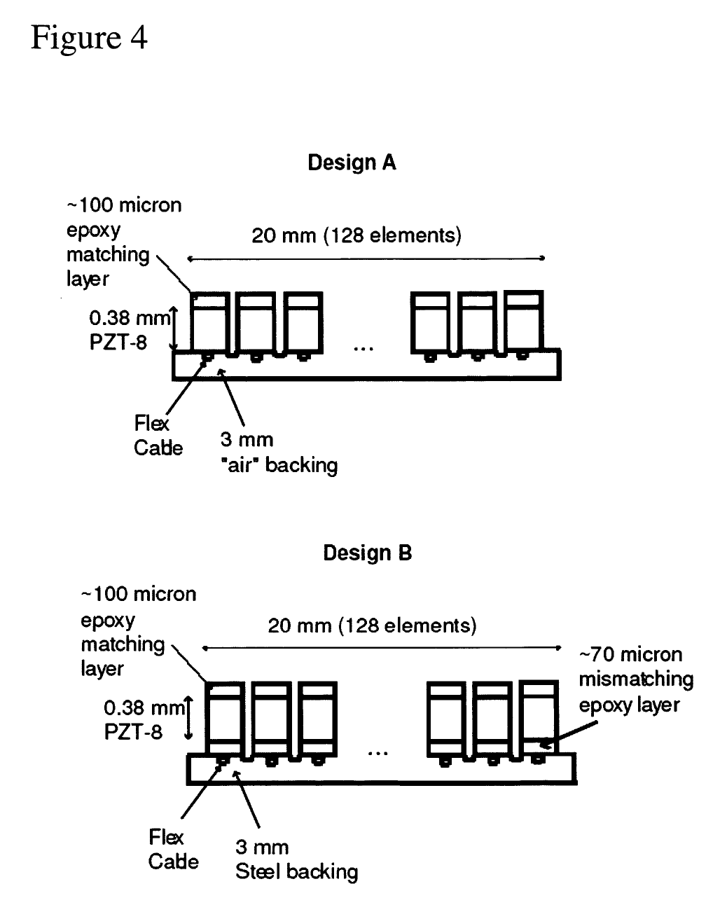

In the design of the dual mode high intensity focused ultrasound array, there are two aspects to consider: 1) selection of the desired materials to meet the desired output power and bandwidth, and 2) treating beam forming issues. Since high intensity arrays usually have a high mechanical Q, it is important to select the appropriate materials to optimize the bandwidth and efficiency for the transducer. Cabling also needs to be considered. As a person of average skill would readily appreciate, several designs can be constructed and evaluated based on bandwidth, efficiency, heating, and cross-coupling to achieve the objective of the present invention. Optimizing transducer designs given a certain set of bandwidth and efficiency criteria is well known in current literature. FIG. 4 shows exemplary designs of transducer array according to the present invention.

The cabling for each transducer could be gold bonds connected to the transducer surface, however these are rather weak and easily broken when attaching matching layers to the surface. Therefore it is preferred to have a flex cable approach in which the transducer is bonded to flex cable (See FIG. 4), pasted on the matching and backing layer, and then diced. The cable traces will extend minimally beneath the array elements as not to affect the acoustic properties of the backing.

As mentioned above, quick ablation times require intensities greater than 200 W/cm² at the focal point (>2 Mpa peak power). Assuming pressure outputs of 1 Mpa from the transducer surface (23 W/cm²), a power gain greater than 16 is required. Since the average radius of the pulmonary vein is 15 mm the array of the present invention establishes this gain over an observation area of roughly 5 mm along the x-axis and 5-15 mm focal depth. Physical movement of the heart wall in this area is on the order of 1 mm, so steering capabilities within a 5 mm by 10 mm area or greater is adequate for the purposes of the present invention. Transducer configurations of greater than 32 elements can accomplish gains of larger than 16 with some degree of grating lobe degradation. To increase gain, one could employ methods such as placing a rough focusing lens on the short (2 mm direction) to create a short knife slice. This could increase the gain at the expense of shorter knife cuts and longer procedure times. An additional approach in increasing gain includes curving the transducer. Grating and side lobes may be an issue in the transducer design, however, these could be solved by using e.g. randomly sized elements, aperiodic weights, or coded excitation.

During a treatment process, the heart is beating. The heart motion can cause problems when depositing ultrasound energy for ablation or imaging. The ostium of the pulmonary veins moves very little (<1 mm over the entire heart cycle), but motion will be disruptive to the ablation process. An average adult has a heart rate of 70 beats per minute; assuming a diastolic period that is a quarter of that cycle, this means the ablation period can be about 100 ms. If the heart is fibrillating during the procedure, the movement of the pulmonary vein is even less since the repetitive stimulus of the atria does not allow heart muscles to recover and contract and relax their full extent. Using an air backing, with acoustic output in excess of 30 W/cm² tissue intensities in excess of 1500 W/cm²could be achieved, which allows one to ablate tissue in under half a second or less. In other words, we are able to image and ablate the tissue in one heart cycle. If these intensities are not great enough to burn in one heart cycle, then one can consider gating to the cardiac cycle and applying multiple doses of energy to necrose tissue. During the time when the pulse is off, there is cooling due to tissue perfusion and convection from the blood, but this should be relatively negligible, since the tissue will retain 65-70% of its temperature during the 1 second when the ablation signal is off.

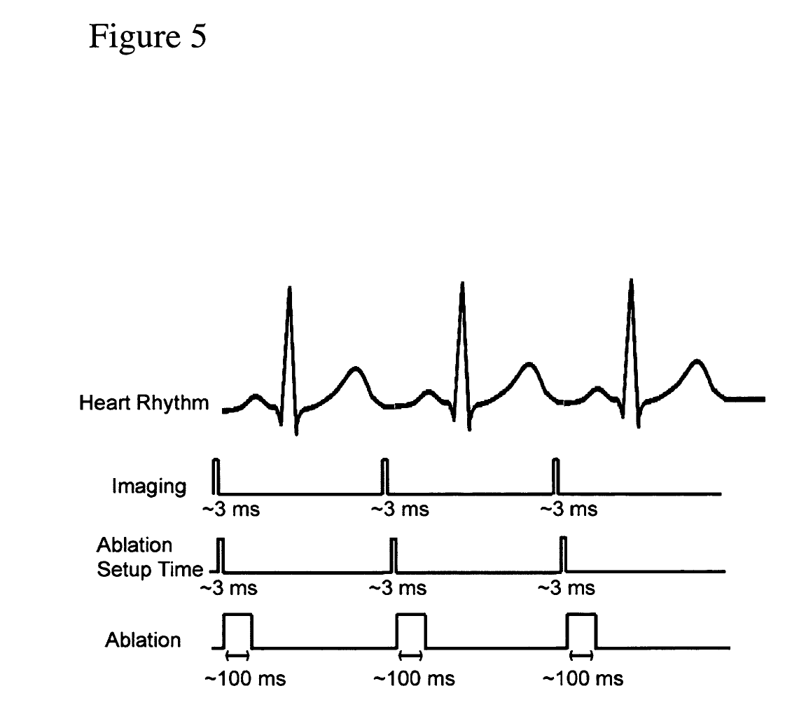

In one aspect imaging can precede ablation at the beginning of diastole (See FIG. 5). For 128 individually fired elements for CSA imaging or regular B-scan imaging and a heart wall at 10-15 mm, it takes about 3 ms to image a 2 cm by 2 cm region. A full phased array scan will require about the same amount of time. Another issue is the time needed to program and focus of the array. Methods of making integrated circuits for driving ultrasonics and fast connections between computer and circuits are well known; such methods could achieve programming rates on the order of 3 ms. Gating can be programmed to every other heart cycle (See FIG. 5) and can be established by a heart cycle monitoring means to monitor the heart cycle or a gating processor. One could integrate an EKG system, a heart cycle monitoring sensor, a blood flow sensor, or a pressure sensor with the catheter. The information regarding the heart cycle is then used to coincide the timing and execution of the processing means within a time frame of a heart cycle (e.g. atrial diastole). If each lesion produces an elliptical burn with 1 mm by 0.1 mm axes and the pulmonary vein is about 15 mm in diameter, about 50-100 burns need to be made. If one burn can be made during 1 heart cycle (See FIG. 5) then the total burning process when fully automated takes 50-100 seconds.

Although the present invention and its advantages have been described in detail, it should be understood that the present invention is not limited to or defined by what is shown or discussed herein. The drawings, description and discussion herein show examples of the invention and provide examples of using the invention. One skilled in the art will realize that implementations of the present invention could be made without departing from the principles, spirit or legal scope of the present invention. Accordingly, the scope of the present invention should be determined by the following claims and their legal equivalents.

1. An apparatus for minimally invasive treatment of an arrhythmia, comprising:
(a) a catheter; and
(b) a dual-mode high intensity focused ultrasound array with an ablation mode and an imaging mode, wherein said array is integrated in the longitudinal direction of said catheter, wherein said array having a side-view with respect to said catheter, and wherein said catheter is small enough to be inserted through a peripheral vein.

2. The apparatus as set forth in claim 1, wherein said catheter has a diameter of up to 4 mm.

3. The apparatus as set forth in claim 1, wherein said catheter has a diameter of up to 3 mm.

4. The apparatus as set forth in claim 1, wherein said array has a length/width ratio of at least 5.

5. The apparatus as set forth in claim 1, wherein said array has a length/width ratio of at least 15.

6. The apparatus as set forth in claim 1, further comprising a stabilization device for said catheter to maintain a distance between said catheter array and the wall of a heart or the wall of a vein.

7. The apparatus as set forth in claim 6, wherein said stabilization device is positioned near or in the pulmonary vein of said heart.

8. The apparatus as set forth in claim 1, wherein said array is a 1-D array or a 2-D array of 32 or more dual-mode ultrasound transducer elements.

9. The apparatus as set forth in claim 1, wherein said array in ablation mode has a power density at the focal area or point of a treatment area of at least 200 W/cm².

10. The apparatus as set forth in claim 1, wherein said catheter is positioned in the left atrium of a heart and said arrhythmia is an atrial fibrillation.

11. The apparatus as set forth in claim 1, further comprising a processing means to:
(i) obtain an image of a region of interest in said imaging mode;
(ii) evaluate said obtained image and determine a treatment area;
(iii) determine ablation parameters for said treatment area;
(iv) focus and steer the elements in said array to said treatment area; and
(v) ablate the tissue of said treatment area in said ablation mode.

12. The apparatus as set forth in claim 11, further comprising a heart cycle monitoring means to monitor the heart cycle and to coincide the timing and execution of said processing means within a time frame of a heart cycle.

13. The apparatus as set forth in claim 11, further comprising a gating processor to gate (i)-(iv).

14. A method for minimally invasive treatment of an arrhythmia, comprising the step of:
(a) inserting through a peripheral vein into a heart a catheter with an integrated dual-mode high intensity focused ultrasound array, wherein said array has an ablation mode and an imaging mode, wherein said array is integrated in the longitudinal direction of said catheter, and wherein said array having a side-view with respect to said catheter;
(b) obtaining an image of a region of interest in said imaging mode;
(c) evaluating said obtained image and determine a treatment area;
(d) determining ablation parameters for said treatment area;
(e) focusing and steering the elements in said array to said treatment area; and
(f) ablating the tissue of said treatment area in said ablation mode.

15. The method as set forth in claim 14, further comprising repeating steps (b)-(f) for another region of interest to create a pattern of ablated tissue.

16. The method as set forth in claim 15, wherein said pattern results in a continuous pattern of ablated tissue.

17. The method as set forth in claim 14, wherein said catheter has a diameter of up to 4 mm.

18. The method as set forth in claim 14, wherein said catheter has a diameter of up to 3 mm.

19. The method as set forth in claim 14, wherein said array has a length/width ratio of at least 5.

20. The method as set forth in claim 14, wherein said array has a length/width ratio of at least 15.

21. The method as set forth in claim 14, further comprising stabilizing said catheter to maintain a distance between said catheter array and the wall of a heart or the wall of a vein.

22. The method as set forth in claim 21, wherein said catheter is stabilized near or in the pulmonary vein of said heart.

23. The method as set forth in claim 14, wherein said array is a 1-D array or a 2-D array of 32 or more dual-mode ultrasound transducer elements.

24. The method as set forth in claim 14, wherein said array in ablation mode has a power density at the focal area or point of a treatment area of at least 200 W/cm².

25. The method as set forth in claim 14, wherein said catheter is positioned in the left atrium of a heart and said arrhythmia is an atrial fibrillation.

26. The method as set forth in claim 14, further comprising gating said steps (b)-(f).

27. The method as set forth in claim 14, further comprising monitoring a heart cycle and coinciding the timing and execution of said steps (b)-(f) within a time frame of said heart cycle.
